(19) United States
(12) Patent Application Publication
(10) Pub. No.: US 2010/0189660 A1
(43) Pub. Date: Jul. 29, 2010

(54) METHODS FOR THE DETECTION OF FATTY-ACYLATED PROTEIN

(75) Inventor: Rami N. Hannoush, San Mateo, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/704,796

(22) Filed: Feb. 12, 2010

Related U.S. Application Data
(60) Provisional application No. 61/207,527, filed on Feb. 14, 2009.

(51) Int. Cl. A61K 49/00; C07C 57/18

(52) U.S. Cl. 424/9.6; 562/598

(57) ABSTRACT

Sensitive, non-radioactive fatty-acyls of Formula I are useful in in vivo methods for detection and cellular imaging of a fatty-acylated substrate (e.g., protein or polypeptide).

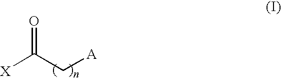

In Formula I the symbols X and A, and the subscript n are as described herein.

These fatty-acyl compounds are can be used, inter alia, for analyzing the lipid composition of proteins in different biological states under various cellular conditions, and serve as a gateway into global lipidomic analysis of cellular proteins.

### CROSS-REFERENCE TO RELATED APPLICATIONS

This application claims priority to U.S. provisional application No. 61/207,527, filed on Feb. 14, 2009, and incorporated herein in its entirety for all purposes.

### BACKGROUND OF INVENTION

Fatty acylation of cellular proteins is vital, controlling protein-protein and protein-membrane interactions. Protein fatty acylation is the covalent attachment of lipids onto proteins. This serves to modulate the proteins' physicochemical properties and biological functions, and to direct their targeting for activation within cells. As such, protein fatty acylation regulates intracellular protein trafficking and sorting, signal transduction pathways and homeostasis (See, Resh, M. D. Trafficking and signaling by fatty-acylated and prenylated proteins. Nat. Chem. Biol. 2, 584-590 (2006); Greaves, J. & Chamberlain, L. H. Palmitoylation dependent protein sorting. J. Cell Biol. 176, 249-254; Zhang, F. L. & Casey, P. J. Protein prenylation: molecular mechanisms and functional consequences. Annu Rev Biochem 65, 241-270 (1996)).

Several classes of protein fatty acylation exist in eukaryotes. These primarily include N-myristoylation and S-palmitoylation (FIG. 1a). Typically, N-myristoylated proteins contain the saturated 14-carbon myristate group bound to an exposed N-terminal glycine residue through a stable amide bond. S-palmitoylation on the other hand comprises the reversible addition of a 16-carbon palmitate or longer fatty acid chains onto cysteine residues via a labile thioester linkage. While S-palmitoylation is dominant in living cells, N-palmitoylation has been identified in Hedgehog and Spitz secreted proteins (See, Pepinsky, R. B. et al. Identification of a palmitic acid-modified form of human Sonic hedgehog. J Biol Chem 273, 14037-45 (1998); Miura, G. I. et al. Palmitoylation of the EGFR ligand Spitz by Rasp increases Spitz activity by restricting its diffusion. Dev Cell 10, 167-76 (2006)) presumably through migration of the palmitoyl group on a cysteine to form an amide linkage.

Despite the critical role of protein fatty acylation in physiology, few methods exist that are highly sensitive for detecting lipid-modified proteins (See, Drisdel, R. C. & Green, W. N. Labeling and quantifying sites of protein palmitoylation. Biotechniques 36, 276-285 (2004); Roth, A. F. et al. Global analysis of protein palmitoylation in yeast. Cell 125, 1003-1013). Traditional methods involve metabolic labeling with radioactive fatty acids (See, Schlesinger, M. J., Magee, A. I. & Schmidt, M. F. Fatty Acid Acylation of Proteins in Cultured Cell. J. Biol. Chem. 255, 10021-10024 (1980)), but they are time consuming as they require extended autoradiographic exposure time, not to mention the hazards of handling radioisotopes. Recently, work describing the metabolic incorporation of fatty acid analogues bearing an azido group and their use to detect fatty acylated proteins by a Staudinger ligation reaction has been presented in the literature. See, Hang, H. C. et al. Chemical probes for the rapid detection of Fatty-acylated proteins in Mammalian cells. J Am Chem Soc 129, 2744-5 (2007); Kostiuk, M. A. et al. Identification of palmitoylated mitochondrial proteins using a bio-orthogonal azido-palmitate analogue. Faseb J 22, 721-32 (2008); Martin, D. D. et al. Rapid detection, discovery, and identification of post-translationally myristoylated proteins during apoptosis using a bio-orthogonal azidomyristate analog. Faseb J 22, 797-806 (2008); and Heal, W. P. et al. Site-specific N-terminal labelling of proteins in vitro and in vivo using N-myristoyl transferase and bioorthogonal ligation chemistry. Chem Commun (Camb), 480-2 (2008). This approach was used for labeling recombinant proteins in bacteria (See, Heal, W. P., Wickramasinghe, S. R., Leatherbarrow, R. J. & Tate, E. W. N-Myristoyl transferase-mediated protein labelling in vivo. Org Biomol Chem 6, 2308-15 (2008)), and for identifying fatty acylated proteins that are localized in mitochondria or posttranslationally modified during apoptosis (See, Kostiuk, M. A. et al. Identification of palmitoylated mitochondrial proteins using a bio-orthogonal azido-palmitate analogue. Faseb J 22, 721-32 (2008); Martin, D. D. et al. Rapid detection, discovery, and identification of post-translationally myristoylated proteins during apoptosis using a bio-orthogonal azidomyristate analog. Faseb J 22, 797-806 (2008)). In view of the above, there remains a need in the art for methods to provide for facile functional and proteomic analysis of protein acylation, in particular in the whole cell environment. The present invention fulfills at least this need.

### SUMMARY OF INVENTION

In one aspect the present invention provides for a method of detecting a fatty-acylated substrate comprising: (i) incubating a fatty acyl of Formula I with an animal cell,

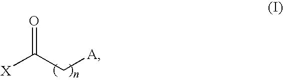

wherein in Formula I the subscript n is an integer from 6 to 15, the symbol A represents an ethynyl group and the symbol X represents —OH or —SCoA, wherein said animal cell comprises a substrate and at least one enzyme capable of attaching I to the substrate, to produce a fatty-acylated substrate; (ii) combining the fatty-acylated substrate from step (i) with an azido tagged labeling group wherein the azido tag undergoes a [3+2] cycloaddition reaction with the A group of the fatty-acylated substrate to produce a labeled fatty-acylated substrate; and (iii) detecting the labeling group on the fatty-acylated substrate; and thereby detecting the fatty-acylated substrate. In certain embodiments, in step (iii), the fatty-acylated substrate is detected in vivo in an animal cell.

The present invention also provides for a method of detecting a fatty-acylated substrate comprising: (i) incubating a fatty-acyl of Formula I with an animal cell

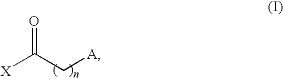

wherein in Formula I the subscript n is an integer from 6 to 15, the symbol A represents an ethynyl group and the symbol X represents —OH or —SCoA, wherein said animal cell comprises a substrate and at least one enzyme capable of attaching I to the substrate, to produce a fatty-acylated substrate; (ii) combining the fatty-acylated substrate from step (i) with an azido tagged labeling group wherein the azido tag undergoes a [3+2] cycloaddition reaction with the A group of the fatty-acylated substrate to produce a labeled fatty-acylated substrate; and (iii) detecting the labeling group on the fatty-acylated substrate in vivo in an animal cell by fluorescence imaging; and thereby detecting the fatty-acylated substrate.

The present invention also provides for the use of a fatty-acyl compound of Formula I in an in vivo assay using an animal cell for the detection of fatty-acylation of a protein or polypeptide,

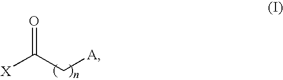

wherein in Formula I the subscript n is an integer from 6 to 15, the symbol A represents an ethynyl group and the symbol X represents —OH or —SCoA, and wherein the detection occurs in an in vivo setting.

### DESCRIPTION OF THE DRAWINGS

FIG. 1 shows a strategy for labeling and imaging of cellular proteins with naturally occurring fatty-acyls and certain compounds of the invention: compounds 1 (C10), 2 (C11), 3 (C13), 4 (C14), 5 (C16) and 6 (C18): (A) Chemical structures of N-myristate and S-palmitate groups covalently attached onto proteins; (B) Exemplary ω-alkynyl fatty-acyls of the invention studied for the invention; (C) Scheme for labeling cellular lipid-modified proteins with exemplary fatty-acyls of Formula I. Synthetic ω-alkynyl fatty-acyls of Formula I were added to cultured cells and metabolically incorporated into acylated proteins (step 1). After work up, the alkynyl group was chemoselectively ligated to azide-tagged biotin or azido-tagged fluorophore by a Cu1-catalyzed alkyne-azide [3+2] cycloaddition reaction. The conjugated proteins were separated by gel electrophoresis and detected by streptavidin-linked horseradish peroxidase (HRP) (route A), or alternatively detected by streptavidin-Alexa488 fluorophore and imaged using fluorescence microscopy (route B).

FIG. 2 show biochemical detection and imaging of lipid-modified proteins: (A) MDCK cells were treated with certain ω-alkynyl fatty-acyl compounds of the invention (100 μM) as indicated for 24 h. lane 1: C10, lane 2: C11, lane 3: C13, lane 4: C14, lane 5: C16, lane 6: C18. Cellular proteome was prepared, reacted with biotin-azide, resolved by gel electrophoresis and detected by western blotting with streptavidin-HRP, using methods as described herein. Asterisks denote bands labeled by treatment with probe but not in DMSO control samples, as judged by increase in intensity or appearance of new bands; (B) In parallel, western blots were treated with 5% hydroxylamine for 72 h before detection with streptavidin-HRP. lane 1: C10, lane 2: C11, lane 3: C13, lane 4: C14, lane 5, C16, lane 6: C18; (C, D, E and F) Fluorescence microscopy of PC3 cells labeled in the absence (C) or presence of ω-alkynyl fatty-acyls C14 (D), C16 (E), and C18 (F). Cells were treated with DMSO or ω-alkynyl fatty-acyls (100 μM) as indicated for 3 h. The cells were then fixed, permeabilized and click reacted with rhodamine-azide and imaged by epifluorescence microscopy. In panels (D), (E), and (F) the fluorescence emission of rhodamine labeled ω-alkynyl fatty-acyls appear as a grey halo surrounding the nuclei which shown as grey circles (Scale bar, 10 μm); (G, H, I) PC3 cells were treated with C14, C16 and C18 ω-alkynyl fatty-acyls (as described above for panels (C-F)) and imaged by confocal microscopy and the imaging results are shown in panels (G), (H) and (I), respectively. All images were acquired the same way using 63× oil objective. The fluorescence along the z-axis is shown on top of each confocal section (Scale bar, 10 μm); (J, K, L) The distribution of lipid-modified proteins in different cellular states can be monitored by fluorescence imaging. Metaphase cells show a distinct distribution of C16-labeled proteins at the plasma membrane and in dense structures around the spindle and throughout the body panel (K). The fluorescence along the z-axis is shown on the left-hand side of panel (K). In cytokinesis, C16-labeled proteins concentrate at the cleavage furrow, the site of cell division panel (L).

FIG. 3 shows labeling and detection of lipid-modified proteins in RAW2647 macrophages (A) and mouse fibroblast L-cells (B). Cells were treated with ω-alkynyl fatty-acyls (100 μM) (lane 1: C10, lane 2: C11, lane 3: C13, lane 4: C14, lane 5: C16, lane 6: C18) for 24 h. Cellular proteome was prepared, reacted with biotin-azide, resolved by gel electrophoresis and detected by western blotting with streptavidin-HRP, using methods as described herein. Asterisks denote bands labeled by treatment with probe but not in DMSO control samples, as judged by increase in intensity or appearance of new bands.

FIG. 4 shows a time-dependent incorporation of C14 (A), C16 (B) and C18 (C) ω-alkynyl fatty-acyl probes into cellular proteins. MDCK cells were treated with ω-alkynyl fatty-acyl probes as indicated. Cellular proteome was prepared, reacted with biotin-azide, resolved by gel electrophoresis and detected by western blotting with streptavidin-HRP, using methods as described herein. Asterisks denote bands labeled by treatment with probe but not in DMSO control samples, as judged by increase in intensity or appearance of new bands.

FIG. 5 shows a dose-dependent incorporation of C14 (A), C16 (B) and C18 (C) ω-alkynyl fatty-acyl probes into cellular proteins. MDCK cells were treated with ω-alkynyl fatty-acyl as indicated. Cellular proteome was prepared, reacted with biotin-azide, resolved by gel electrophoresis and detected by western blotting with streptavidin-HRP, using methods as described herein. Asterisks denote bands labeled by treatment with probe but not in DMSO control samples, as judged by increase in intensity or appearance of new bands.

FIG. 6 shows the specificity of incorporation of ω-alkynyl fatty-acyls: (A) Inhibition of C14 labeling in the presence of cycloheximide. MDCK cells were treated with C14 ω-alkynyl fatty acid (100 μM) in the presence or absence cycloheximide (100 μg/ml) for 5 h. Cellular proteome was prepared, reacted with biotin-azide, resolved by gel electrophoresis and detected by western blotting with streptavidin-HRP, as described using methods described herein. Asterisks denote bands labeled by treatment with probe but not in DMSO control; (B, C) Dose-dependent competition of C14 and C16 ω-alkynyl fatty acids with myristic (MA) and palmitic acids (PA), respectively. MDCK cells were treated with ω-alkynyl fatty acid probes as indicated in the presence of increasing concentration of myristic (MA) and palmitic acids (PA). Samples were processed as described herein.

FIG. 7 shows fluorescence microscopy data of cellular proteins labeled with ω-alkynyl fatty-acyls in PC3 prostate cancer cells. Cells were treated with DMSO (A) or 100 μM of C10 (B), C13 (C), C14 (D), C16 (E), C18 (F) for 24 h. Cells were then fixed, permeabilized and click reacted with biotin-azide followed with treatment with streptavidin-conjugated Alexa488 and (optionally Hoechst stain for nuclei staining) and imaged using epifluorescence microscopy technique as described herein.

FIG. 8 shows fluorescence microscopy data of cellular proteins labeled with ω-alkynyl fatty-acyls in mouse fibroblast L-cells. Cells were treated with DMSO (A) or 100 μM of C10 (B), C11 (C), C13 (D), C14 (E), C16 (F), C18 (G) for 24 h. Cells were then fixed, permeabilized and click reacted with biotin-azide followed treatment with streptavidin-conjugated Alexa488 and (optionally Hoechst stain for nuclei staining) and imaged using epifluorescence microscopy technique as described herein.

FIG. 9 shows fluorescence microscopy data of cellular proteins labeled with ω-alkynyl fatty-acyls in RAW2647 macrophages. Cells were treated with DMSO (A) or 100 μM of C10 (B), C11 (C), C13 (D), C14 (E), C16 (F), C18 (G) for 24 h. Cells were then fixed, permeabilized and click reacted with biotin-azide followed with treatment with streptavidin-conjugated Alexa488 and (optionally Hoechst stain for nuclei staining) and imaged by epifluorescence microscopy as described in herein.

### DETAILED DESCRIPTION OF THE INVENTION

### Definitions

As used herein, the terms “protein” and “polypeptide” can be used interchangeably throughout the application and mean at least two covalently attached amino acids, which includes proteins, polypeptides, oligopeptides and peptides. The protein can be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradation.

As used herein, the term “substrate” refers to a substance that is acted upon by an enzyme.

As used herein, the term “enzyme” refers to a biomolecule, which is typically a protein that can catalyze chemical reactions.

As used herein, a “label” or “labeling group” is meant a molecule that can be directly (i.e., a primary label) or indirectly (i.e., a secondary label) detected, for example, a label can be visualized and/or measured or otherwise identified so that its presence or absence can be known. As will be appreciated by those in the art, the manner in which this is done will depend on the label. Suitable labeling groups that can be used in the present invention include primary detectable labels, such as for example fluorescent labels, FRET energy donors, label enzymes, among others, and secondary labels, such as a member of a binding pair, among others.

As used herein, a “label enzyme” is meant as an enzyme which may be reacted in the presence of a label enzyme substrate to produce a detectable product. Suitable label enzymes for use in the present invention include, but are not limited to, horseradish peroxidase, alkaline phosphatase, and glucose oxidase. Methods for the use of such substrates are well known in the art and are also described herein. The presence of the label enzyme is generally revealed through the enzyme's catalysis of a reaction with a label enzyme substrate, producing an identifiable product. Such products may opaque, such as the reaction of horseradish peroxidase with tetramethyl benzedine, and may have a variety of colors. Other label enzyme substrates, such as Luminol (available from Thermo Fisher Scientific), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

As used herein, “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 2002 Molecular Probes Handbook Ninth Edition by Richard P. Haugland, hereby expressly incorporated by reference. Suitable fluorescent labels also include, but are not limited to, green fluorescent protein (GFP; Chalfie, et al., Science 263(5148):802-805 (Feb. 11, 1994); and EGFP; Clontech-Genbank Accession Number U55762), blue fluorescent protein (BFP; 1. Evrogen Inc. Miklukho-Maklaya str, 16/10, 117997, Moscow, Russia; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; Clontech Laboratories, Inc., 1290 Terra Bella Avenue, Mountain View, Calif. 94043, USA), luciferase (Ichiki, et al., J. Immunol. 150(12):5408-5417 (1993)), beta-galactosidase; (Nolan, et al., Proc Natl Acad Sci USA 85(8):2603-2607 (April 1988)), and Renilla; U.S. Pat. Nos. 5,292,658; 5,418,155; 5,683,888; 5,741,668; 5,777,079; 5,804,387; 5,874,304; 5,876,995; and 5,925,558) All of the above-cited references are expressly incorporated herein by reference.

In addition, labels may be indirectly detected, and as such, a label group can be, for example, a member of a binding pair. As used herein a “member of a binding pair” is meant one of a first and a second moiety, wherein said first and said second moiety have a specific binding affinity for each other. Suitable binding pairs for use in the invention include, but are not limited to, biotin/avidin (or biotin/streptavidin), antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine/anti-rhodamine) and calmodulin binding protein (CBP)/calmodulin. Other suitable binding pairs include polypeptides such as the FLAG-peptide (Hopp et al., BioTechnology, 6:1204-1210 (1988)); the KT3 epitope peptide (Martin et al., Science, 255:192-194 (1992)); tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA 87:6393-6397 (1990)) and the antibodies each thereto.

As will be appreciated by those in the art, a complementary member of one binding pair can also be a complementary member of another binding pair. For example, an antigen (first moiety) may bind to a first antibody (second moiety) which can, in turn, be an antigen for a second antibody (third moiety). It will be farther appreciated that such a circumstance allows indirect binding of a first moiety and a third moiety via an intermediary second moiety that is a member of a binding pair complementary to each.

As will be appreciated by those in the art, labeling group can comprise a member of a binding pair, as described above. It will further be appreciated that this allows a compound (e.g., a fatty-acylated substrate) to be indirectly labeled upon the binding of a member of a binding pair, e.g. a biotin moiety. Attaching one member of a binding pair to a substrate (e.g., a fatty-acylated substrate), such member of a binding pair having a complementary binding partner, e.g., streptavidin, is referred to herein as “indirect labeling.”

The term “alkylene” means a divalent radical derived from an alkyl, as exemplified by —CH₂CH₂ CH₂CH₂₋ and —CF₂CF₂₋. Typically, an alkyl (or alkylene) group will have from 1 to 24 carbon atoms, with those groups having 10 or fewer carbon atoms being preferred in the present invention. For clarity the term “alkyl” means a straight or branched chain hydrocarbon radical and halogenated variants, having the number of carbon atoms designated (e.g., C₁₋₆ means one to six carbons).

The term “heteroalkylene” means a divalent radical derived from heteroalkyl, as exemplified by —O—CH₂₋CH₂₋CH₂—CH₂₋O—, —O—CH₂, —CH₂₋O—, —CH₂₋CH₂₋S—CH₂CH₂₋ and —CH₂₋S—CH₂₋CH₂₋NH—CH₂₋, —O—CH₂₋CH═CH—, —CH₂₋CH═C(H)CH₂₋O—CH₂₋, —O—CH₂₋CHÿCH—, —S—CH₂₋CÿC—, —CF₂₋O—. For clarity, the term “heteroalkyl,” means a stable straight or branched chain hydrocarbon radical, consisting of the stated number of carbon atoms and from one to three heteroatoms selected from the group consisting of O, N, Si and S, and wherein the nitrogen and sulfur atoms can optionally be oxidized and the nitrogen heteroatom can optionally be quaternized. As used herein, the term “heteroalkylene” also refers to mono- and poly-halogenated variants.

### Embodiments of the Invention

There remains a need in the art for methods to provide for facile functional and proteomic analysis of protein fatty acylation, in particular in the whole cell environment. The present invention fulfills this need by providing for the use of non-radioactive alkyne containing fatty-acyls of Formula I:

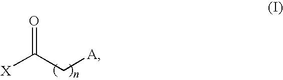

in which in Formula I the subscript n is an integer from 6 to 15, the symbol A represents an ethynyl group and the symbol X represents —OH or —SCoA, which can be metabolically incorporated onto substrates, such as proteins and polypeptides into the cellular environment.

The compounds of Formula I find utility at least for the detection and visualization of fatty-acylated substrates in animal cells.

As used herein the abbreviation SCoA represents the coenzyme A group having the structure

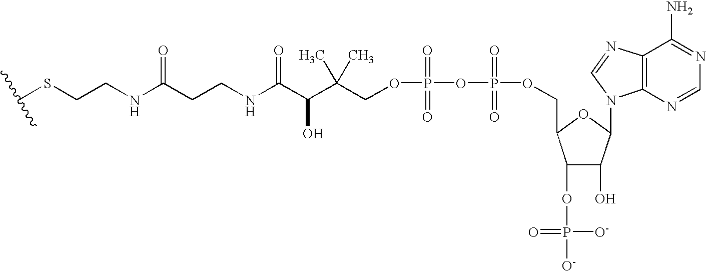

in which the wavy line [symbol] denotes the point of attachment of the coenzyme A group to the remainder of the compound of Formula I.

Surprisingly, Applicants have discovered that alkyne containing fatty-acyl of Formula I can be used for the fatty-acylation of a substrate such as a protein or peptide upon incubation in an in vivo setting, in an animal cell (in one embodiment, a mammalian cell, and in another embodiment, in a cancer cell), wherein the animal cell, or each embodiment thereof, comprises an enzyme capable of catalyzing the fatty-acylation of the substrate with a compound of Formula I. Advantageously, a compound of Formula I is highly suitable for this purpose. Without being bound by any particular theory, the inventor believes that the alkyne group on the fatty-acyl carbon chain of Formula I maintains the hydrophobicity of the fatty-acyl chain to result in its minimal interference with the physicochemical properties of the fatty-acyl chain and its interactions. Moreover, once an alkyne containing fatty-acyl of Formula I is attached to a substrate, such as a protein or peptide, the alkynyl group thereon is metabolically inert but sufficiently reactive under appropriate chemical conditions and, as such, the alkyne moiety can be used as a point of attachment for a labeling group comprising an azido tag.

A label or labeling group comprising an azido tagging moiety can also comprise a linking group which connects the label with the azido tagged moiety. In one embodiment, the labeling group is directly attached to an azido tagged moiety. In another embodiment, a linking group is attached to an azido moiety through a linking group. Typically, a linking group or linker is a relatively short non-reactive coupling moiety that is used to tether an azido moiety with a labeling group, such as for example, a C₁₋₁₂ alkylene linker or a C₁₋₁₂ heteroalkylene linker, such as those provided in the examples below.

A number of azido tagged labeling groups are available for purchase through commercial suppliers. Invitrogen (Carlsbad, Calif.) sells a number of azido tagged labels as “Click Chemistry Reagents.” In particular the Click-iT™ azide reagents are suitable for use in the invention. These include: AlexaFluor®488 azide—(Alexa Fluor® 488 5-carboxamido-(6-azidohexanyl), bis(triethylammonium salt)), catalog number A10266; AlexaFluor®594 azide—(Alexa Fluor® 594 carboxamido-(6-azidohexanyl), triethylammonium salt), catalog number A10270; AlexaFluor®647 azide, catalog number A10277; biotin azide—PEG4 carboxamide-6-azidohexanyl biotin, catalog number B10184; Oregon Green®488 azide—(Oregon Green® 6-carboxamido-(6-azidohexanyl), triethylammonium salt), catalog number O10180; tetramethylrhodamine azide—tetramethylrhodamine 5-carboxamido-(6-azidohexanyl)), catalog number T10182. Other azido tagged labeling groups are known to on skilled in the art which can be prepared by known synthetic methods or can be available from commercial sources.

A particularly useful method for the attachment of a labeling group to a fatty-acylated substrate, is to use a copper I catalyzed variation of the Huisgen [3+2] cycloaddition reaction between an alkyne and azido-tagged group developed by Sharpless et al. as described in U.S. Pat. No. 7,375,234, which is incorporated herein by reference for this teaching, and is outlined below. Sharpless et al. have coined this variation of the Huisgen [3+2] cycloaddition reaction as the “click reaction.” The click reaction used in the invention is illustrated in Scheme 1 below: a fatty-acylated substrate A1 of the invention comprising an ethynyl,

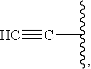

group and an azido-tagged labeling group A2, when combined, provides for a labeled fatty-acyl substrate A3¹ and A3², with the A3¹ isomer usually predominating. When it is described in the application that an alkynyl containing substrate and an azido-tagged moiety “undergoes a [3+2] cycloaddition reaction”, it is meant that the alkynyl group and the azido group react with each other in a cycloaddition reaction as shown in Scheme 1 below and the product of such a reaction contains a triazole functional group. In certain embodiments, a copper (I) reagent is added to catalyze the [3+2] cycloaddition reaction. In one embodiment, the substrate is a protein or polypeptide. In another embodiment, the reaction is performed in an in vivo setting. In one embodiment, the azido tagged labeling group is biotin azide (Invitrogen catalog number B10184). In another embodiment, the azido tagged labeling group is tetramethylrhodamine azide (Invitrogen catalog number T10182). In another embodiment, the azido tagged labeling group is rhodamine-azide (see, Speers, A. E. & Cravatt, B. F. Profiling enzyme activities in vivo using click chemistry methods. Chem. Biol. 11, 535-546 (2004)).

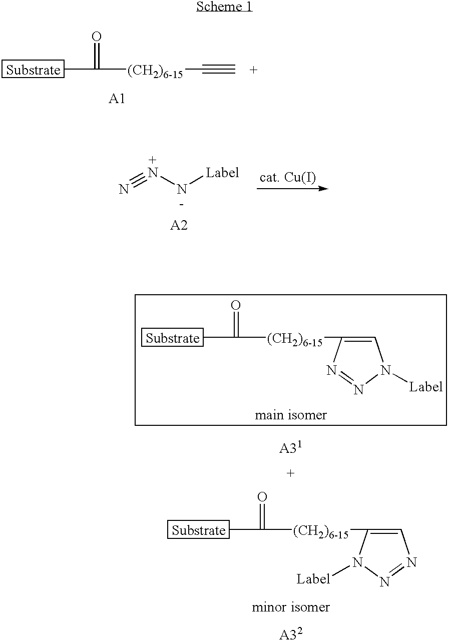

After the attachment of the labeling group to a substrate (e.g., a protein or polypeptide) that has been fatty-acylated with a compound of Formula I, it is possible to detect the fatty-acylated substrate product by detection of the labeling group thereon. Detection of the labeling group that is attached to a substrate is performed using methods and reagents well known to those skilled in the art, including, but not limited to, fluorescence imaging, western blotting, mass spectrometry, and fluorescence spectroscopy. Optionally, the labeling group attached to a fatty-acylated substrate (as exemplified in Scheme 1 as A3¹ and A3²) is a member of a binding pair (e.g., biotin azide) and prior to detection, the labeled fatty-acylated substrate is incubated a compound comprising the complementary member of the binding pair and is linked to another label, such as, for example, a fluorescent group, a label enzyme, among others (e.g., streptavidin linked fluorophores, such as those available from Invitrogen (Carlsbad, Calif.) including streptavidin linked: AlexaFluor®488 cat. no. S32354; tetramethylrhodamine cat. no. S870; fluorescein cat. no. S869, rhodamine B cat. no. S871; AlexaFluor® 660 cat. no. S21377, among others), which is detected, to thereby detect the fatty-acylated protein.

A preferred method of detection used for the invention is through the detection of fluorescence emission. In one embodiment, fluorescence emission from the complex can be visualized with a variety of fluorescence imaging techniques, including, but not limited to, ordinary light or fluorescence microscopy (epifluorescence microscopy), confocal laser-scanning microscopy, and flow cytometry, optionally using image deconvolution algorithms. Three-dimensional imaging resolution techniques in confocal microscopy utilize knowledge of the microscope's point spread function (image of a point source) to place out-of-focus light in its proper perspective. Substrates labeled with different labeling groups can be optionally resolved spatially, chronologically, by size, or using detectably different spectral characteristics (including excitation and emission maxima, fluorescence intensity, fluorescence lifetime, fluorescence polarization, fluorescence photobleaching rates, or combinations thereof), or by combinations of these attributes. In one embodiment, the method of detection used for the invention is fluorescence imaging.

Another preferred method of detection used for the invention is western blotting.

Inventor discloses herein a method for detecting substrates that have been fatty-acylated with compounds of Formula I in an in vivo setting (e.g., in an animal cell, such as a mammalian cell, or cancer cell); and further discloses the use of compounds of Formula I in an in vivo assay setting as described below. For example, the compounds of Formula I find utility as probes to be used for routine biochemical detection of protein fatty-acylation, such as for example, palmitoylation and myristoylation, of substrates in animal cells, and for fluorescence imaging of global protein fatty acylation in animal cells without the need for radioactive probes. The compounds of Formula I and the methods described herein will be useful in the analysis of cellular processes in biological systems involving fatty-acylation and in the purification of fatty-acylated cellular substrates, such utility including, for example, (a). for assessing the lipidation status of any specific protein of interest; (b) for enriching trace proteins by label incorporation and facilitating separation of proteins that are otherwise difficult to immunoprecipitate with antibodies; (c) for the identification of new acylated proteins; (d) as a diagnostic reporter in imaging assays for analyzing fatty-acylation of substrates, e.g., protein, in response to drugs like N-myristoyltransferase and palmitoyltransferase inhibitors; (e) screening candidate modulators of acyl-transferases; and (f) for the site-specific tagging of antibodies.

Accordingly, in one aspect, in a first embodiment, the invention provides for a method of detecting a fatty-acylated substrate comprising:
i. incubating a fatty acyl of Formula I with an animal cell,

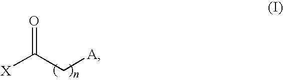

wherein in Formula I the subscript n is an integer from 6 to 15, the symbol A represents an ethynyl group and the symbol X represents —OH or —SCoA,
wherein said animal cell comprises a substrate and at least one enzyme capable of attaching I to the substrate, to produce a fatty-acylated substrate;
ii. combining the fatty-acylated substrate from step (i) with an azido tagged labeling group wherein the azido tag undergoes a [3+2] cycloaddition reaction with the A group on the fatty-acylated substrate to produce a labeled fatty-acylated substrate; and
iii. detecting the labeling group on the fatty-acylated substrate; and thereby detecting the fatty-acylated substrate.

In a second embodiment, the present invention provides for a method of detecting a fatty-acylated substrate comprising:
i. incubating a fatty acid of Formula I with an animal cell

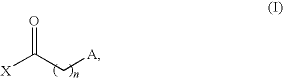

wherein in Formula I the subscript n is an integer from 6 to 15, the symbol A represents an ethynyl group and the symbol X represents —OH or —SCoA,
wherein said animal cell comprises a substrate and at least one enzyme capable of attaching I to the substrate, to produce a fatty-acylated substrate;
ii. combining the fatty-acylated substrate from step i with an azido tagged labeling group wherein the azido tag undergoes a [3+2] cycloaddition reaction with the A group on the fatty-acylated substrate to produce a labeled fatty-acylated substrate; and
iii. detecting the labeling group on the fatty-acylated substrate in vivo in an animal cell by fluorescence imaging; and thereby detecting the fatty-acylated substrate.

In another embodiment, in certain aspects of the first or second embodiment, the method is performed in a mammalian cell.

In another embodiment, within certain aspects of the first or second embodiment, the cell is a cancer cell.

In another embodiment, in certain aspects of the first or second embodiment, the enzyme is acyltransferase. In certain aspects, the enzyme is selected from the group consisting of N-myristoyltransferase, S-acyltransferase and S-palmitoyltransferase.

In another embodiment, within certain aspects of the first or second embodiment, in Formula I the subscript n is an integer from 7 to 14. In certain aspects, the subscript n is an integer selected from the group consisting of 7, 8, 10, 11 and 13. In certain other aspects, the subscript n is the integer 11 or 13.

In another embodiment, in certain aspects of the first or second embodiment, in Formula I X is —OH.

In another embodiment, in certain aspects of the first or second embodiment, in Formula I X is —SCoA.

In another embodiment, in certain aspects of the first or second embodiment, the substrate is a protein or polypeptide.

In another embodiment, in certain aspects of the first embodiment, the labeling group is selected from the group consisting of a label enzyme and a fluorescent labeling group. In certain aspects, the labeling group is rhodamine azide. In certain aspects, the labeling group is biotin azide.

In another embodiment, in certain aspects of the first or second embodiment, the labeling group comprises a member of a binding pair. In certain aspects of this embodiment, in the method between steps ii and iii is a step of treating the labeled fatty-acylated substrate produced from step ii with a detectable labeling group comprising a complementary member of said binding pair, and wherein said complementary member of said binding pair binds to the labeling group of said labeled fatty-acylated substrate produced from step ii. In certain aspects of this embodiment, the complementary member of said binding pair is streptavidin linked to a fluorophore. In certain aspects of this embodiment, the complementary member of said binding pair is streptavidin linked AlexaFluor 488.

In another embodiment, in certain aspects of the first embodiments, in step iii of the method the labeled fatty-acylated substrate is detected by western blotting, mass spectrometry or fluorescence imaging. In certain aspects, the labeled fatty-acylated substrate is detected by fluorescence imaging.

In another embodiment, in certain aspects of the first embodiment, the labeling group is detected in vivo in a mammalian cell, or cancer cell.

In another aspect, the present invention provides, for the use of a fatty-acyl compound of Formula I in an in vivo assay in an animal cell for the detection of fatty-acylation of a protein or polypeptide,

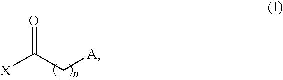

wherein in Formula I the subscript n is an integer from 6 to 15, the symbol A represents an ethynyl group and the symbol X represents —OH or —ScoA, and wherein the detection occurs in an in vivo setting. In certain aspects of the nineteenth embodiment, the assay is performed using mammalian cells. In certain aspects of this embodiment, the assay is performed using cancer cells. In certain aspects of this embodiment, in Formula I, the subscript n is an integer from 7 to 14. In certain aspects of this embodiment, the subscript n is an integer selected from the group consisting of 7, 8, 10, 11 and 13. In certain aspects of this embodiment, the subscript n is an integer selected 11 or 13.

Synthesis of Compounds

As shown in Scheme 2 below, compounds of Formula I can be synthesized, for example, from the corresponding alcohols having internal alkynes (B1) via a zipper reaction (See, Brown, C. A. & Yamashita, A. Saline hydrides and superbases in organic reactions. IX. Acetylene zipper. Exceptionally facile contrathermodynamic multipositional isomeriazation of alkynes with potassium 3-aminopropylamide. J. Am. Chem. Soc. 97, 891-892 (1975)) which results in the isomerization of an internal alkyne to a terminal alkyne (B2). This was followed by Jones oxidation to provide for fatty-acyls (B3). Coupling of fatty-acyls (B3) with coenzyme A via an activated acyl derivatives of (B3) (which can by prepared by synthetic methods described in Mishra, P. K. and Drueckhammer, D. G. Coenzyme A Analogues and Derivatives: Synthesis and Applications as mechanistic Probes of Coenzyme A Ester-utilizing Enzymes. Chem. Rev. 100(9) 3283-3310) should provide the coenzyme A derivative (B4). In compounds B1, B2, B3 and B4, the subscripts m and n are independently an integer between 0 and 13 provided that the combined values of m and n within each compound is less than or equal to 13.

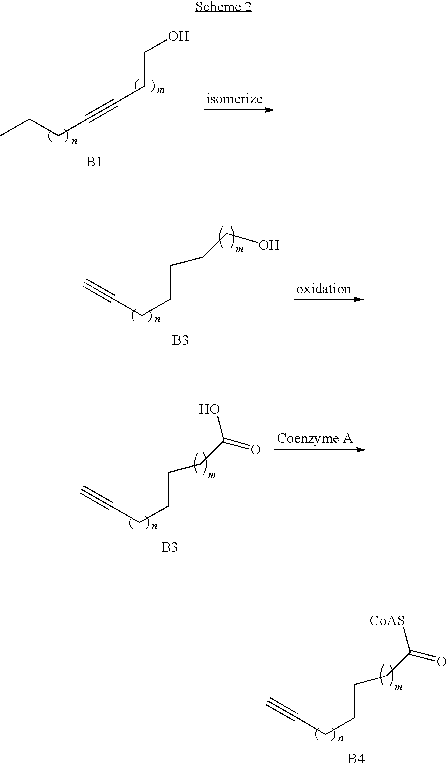

A labeling group (e.g, D3) comprising an azido moiety attached through a linker can be prepared following the synthetic method as outlined below in Scheme 3.

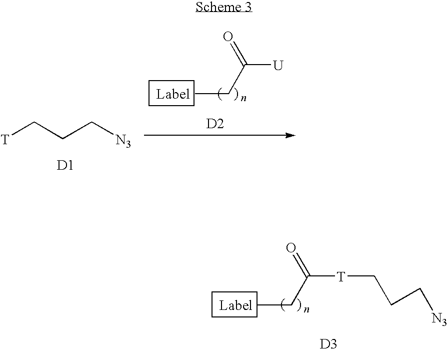

For example, a linker can already comprise an azido tag at one terminus and further contain at least one functional group (e.g., a nucleophile such as an amino group or hydroxy group represented as “T” in compound (D1)) to facilitate attachment of the azido tag to a labeling group (e.g., D2) comprising a suitable leaving group “U” functional group such as halide or triflate or carboxyl derivative (e.g., —CC(O)CCl₃). Such reactions can be performed, typically in an aprotic solvent, such as dimethylformamide, in the presence of a weak base, such as triethylamine, for example. In Scheme 3, a label can be a primary or secondary label, such as for example, rhodamine, biotin, among others.

Alternatively, the linker group can have at least two functional groups, which are used to attach a functionalized labeling group and to a functionalized azido tag, for example. The linker can also be a polymer. In certain cases, an azido tagged labeling group does not contain a linker. In this instance, the labeling group is directly attached to the azido tag. The labeling group and azido tag may be attached in a variety of ways, including those listed above, so long as the manner of attachment does not significantly alter the functional purpose of the labeling group.

As generally outlined above, a linker group to which an azido tag is attached, can be functionalized to facilitate covalent attachment, to a labeling group: other suitable functional groups, including, but not limited to, isothiocyanate groups, amino groups, haloacetyl groups, maleimides, succinimidyl esters, and sulfonyl halides, all of which may be used to covalently attach the azido tag to a labeling group. It is expected that one skilled in the art would understand that in the instance that an azido tagged linker group is functionalize with a T group that is an electrophilic group, e.g., a maleimide, then the labeling group should be functionalized with a U group that is a suitably reactive nucleophilic group. For example, Invitrogen (Carlsbad, Calif.) sells a PEG linker, having an azido group attached on one terminus of the linker and further having a succinimidyl ester functional group attached on the other terminus of the PEG linker “(azido polyethylene glycol (PEG4), succinimidyl ester”, catalog number A10280. This compound could be attached to a labeling group comprising an amino functional group for attachment. More generally, the choice of the functional group on the linker will depend on the site of attachment to either a linker, as outlined above or a labeling group.

The following examples are provided merely for the purpose of illustrating the invention and should no way be construed as to limit the scope of the claimed invention.

### EXAMPLES

### Example 1

### Metabolic Incorporation of Fatty-Acyls of Formula I in to Cellular Proteins

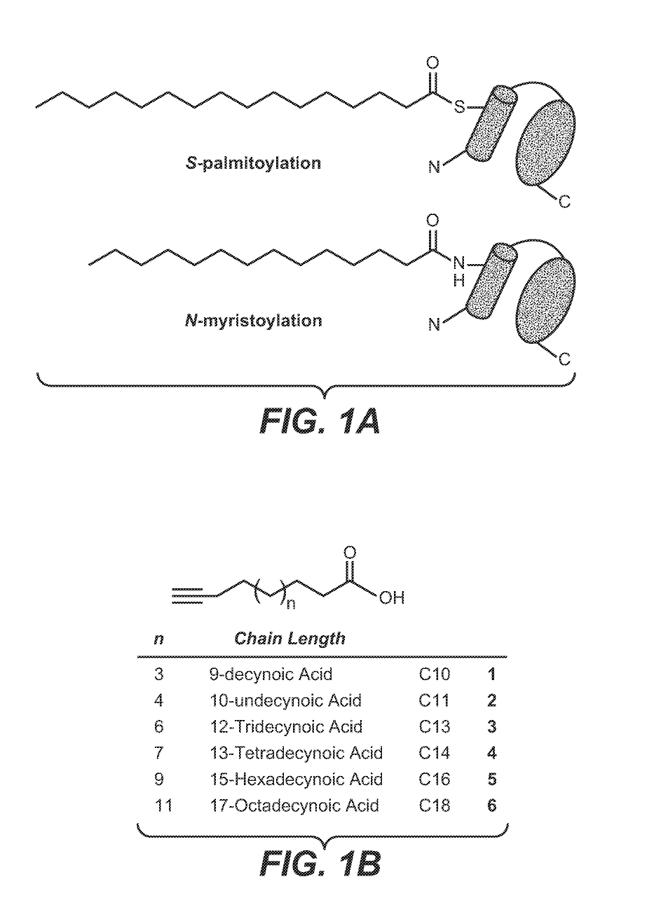

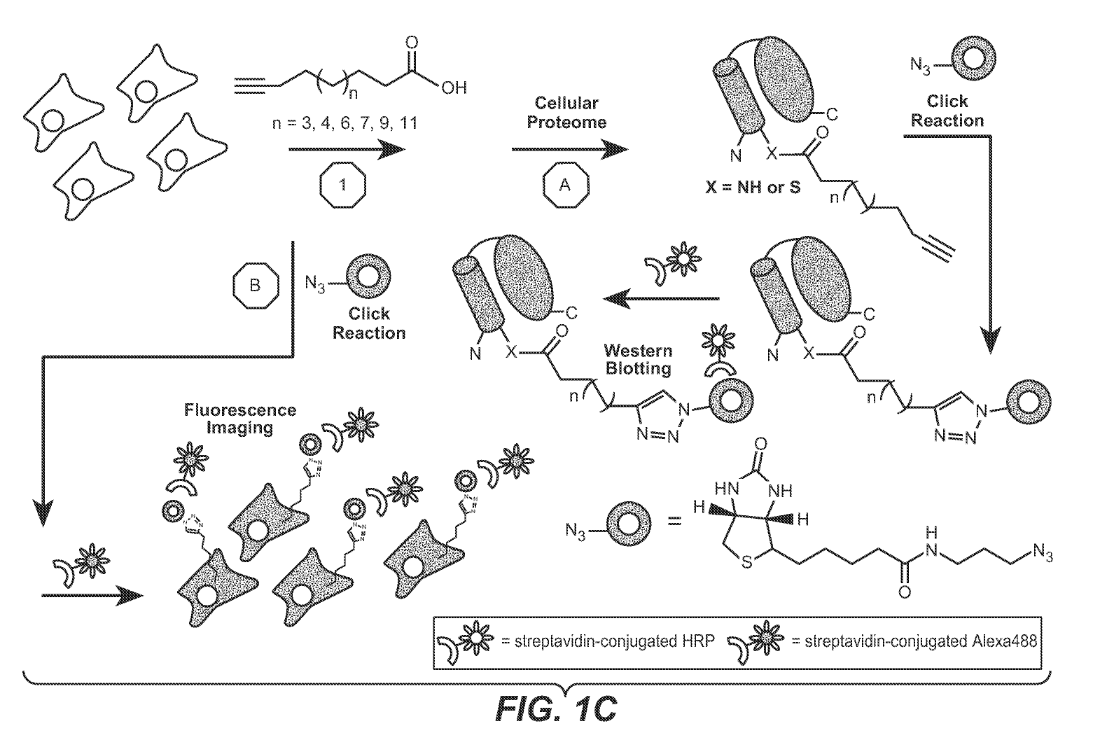

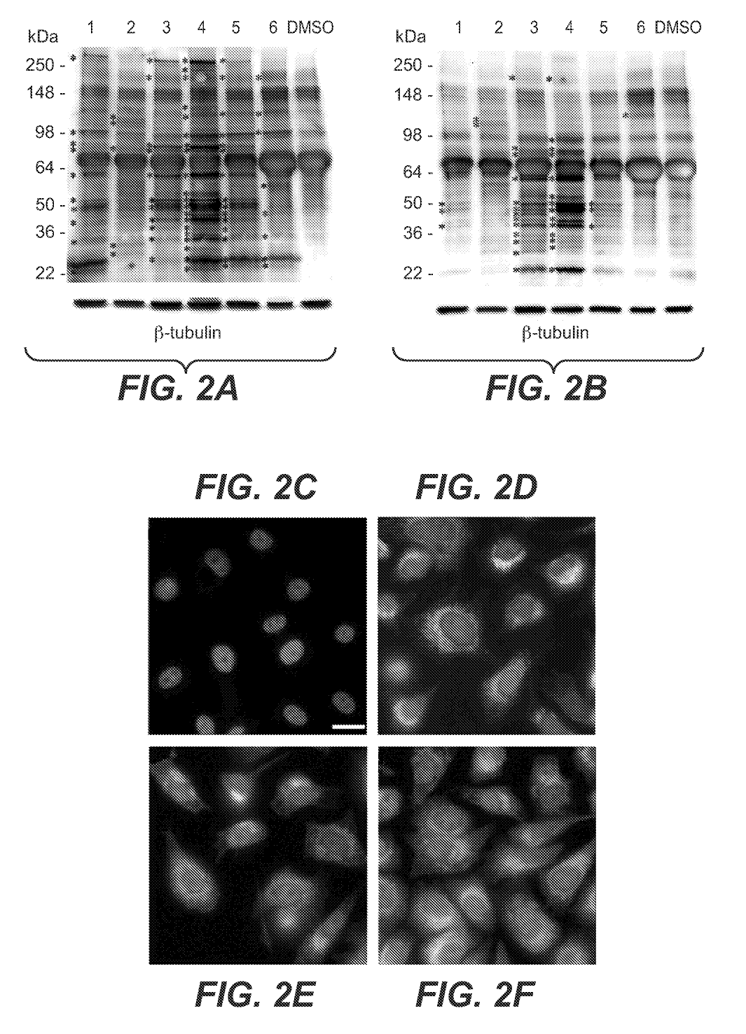

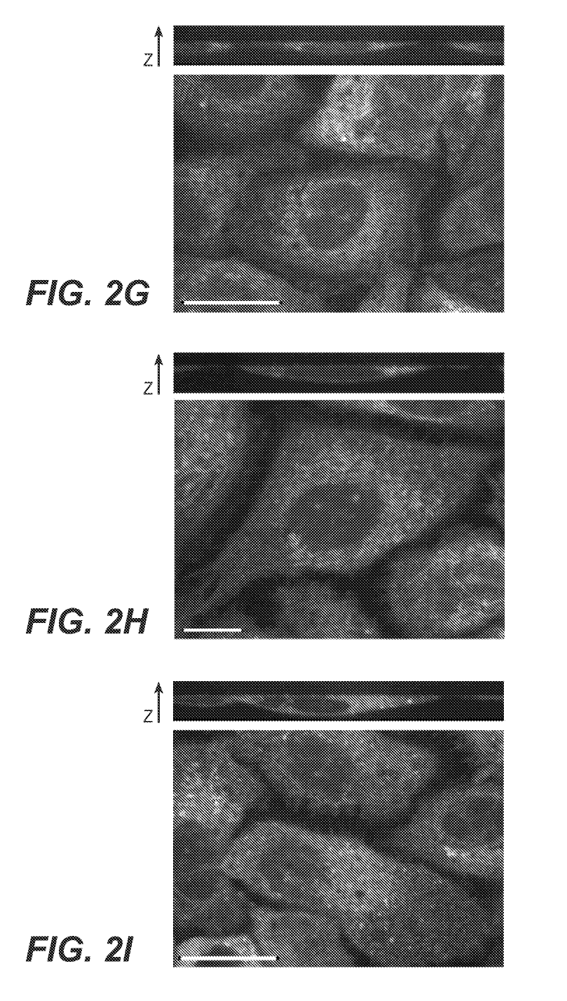

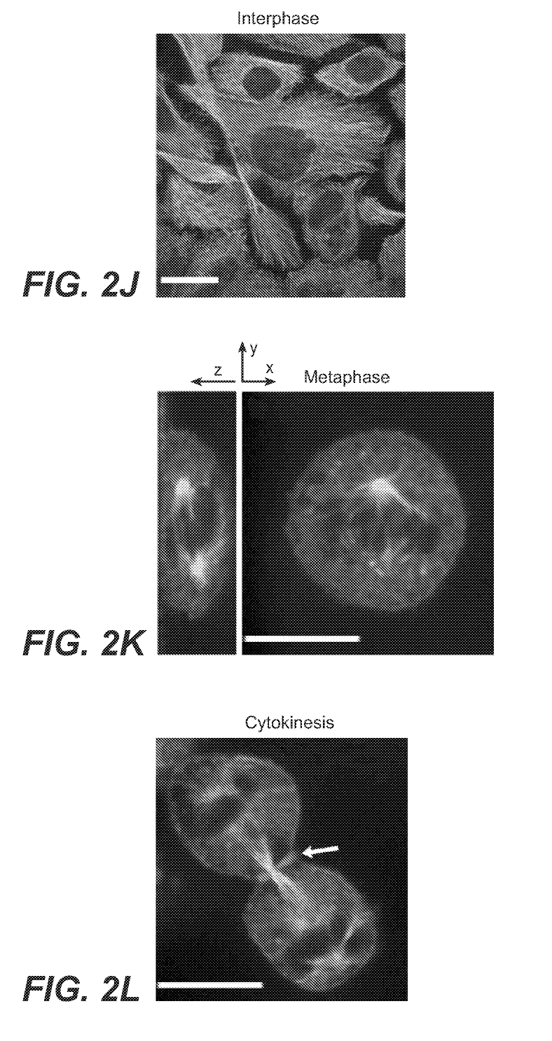

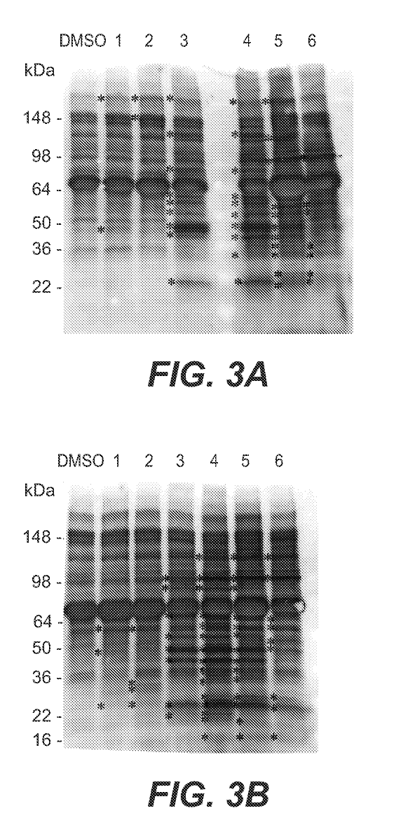

To demonstrate that the synthetic fatty-acyls of Formula I were metabolically incorporated onto cellular proteins, ω-alkynyl fatty-acyls with C10 (1), C11 (2), C13 (3), C14 (4), C16 (5), and C18 (6) carbon atoms (See, FIG. 1B) were exogenously added to cultured MDCK cells and incubated for 24 h. Upon preparing the cellular proteome, the alkynyl group incorporated onto acylated proteins was chemoselectively ligated to azide-tagged biotin (for synthesis see Example 2) or fluorophore by a Cu(I)-catalyzed Huisgen alkyne-azide cycloaddition reaction (See, Wang, Q. et al. J. Am. Chem. Soc. 125, 3192-3193 (2003)) (FIG. 1C). The conjugated proteins were separated by gel electrophoresis and analyzed by Western blot using streptavidin-linked horseradish peroxidase (FIG. 2A). Various proteins were labeled depending on the carbon chain length, with C13, C14 and C16 exhibiting the highest degree of protein incorporation. This was reasonable considering that the majority of protein lipid modifications in cells comprise myristoylation and palmitoylation. Furthermore, the ω-alkynyl fatty-acyls were efficiently uptaken and metabolically incorporated into other cell lines such as RAW2647 macrophages and mouse L-cells (FIG. 3), demonstrating the versatility of these probes.

To demonstrate the specificity of metabolic incorporation, the alkyne-labeled proteins from MDCK cells were treated with hydroxylamine (FIG. 2B), which selectively removes fatty-acyls attached to proteins via thioester but not amide bonds (see, Drisdel, R. C. & Green, W. N. Labeling and quantifying sites of protein palmitoylation. Biotechniques 36, 276-285 (2004)). The ω-alkynyl fatty acyl with 16 carbon atoms exhibits substantial sensitivity to hydroxylamine, and hence is predominantly attached via thioester linkages. On the other hand, the C13 and C14-carbon fatty-acyl chains predominantly were incorporated via amide bonds as inferred by their resistance to hydroxylamine treatment. These experiments validate the utility of C14 and C16 ω-alkynyl fatty-acyls as probes for protein myristoylation and palmitoylation, respectively. The experiments also demonstrate that C10, C11 and C18 predominantly attach via thioester bonds (FIG. 2B), and hence serve as probes of S-acylation as well.

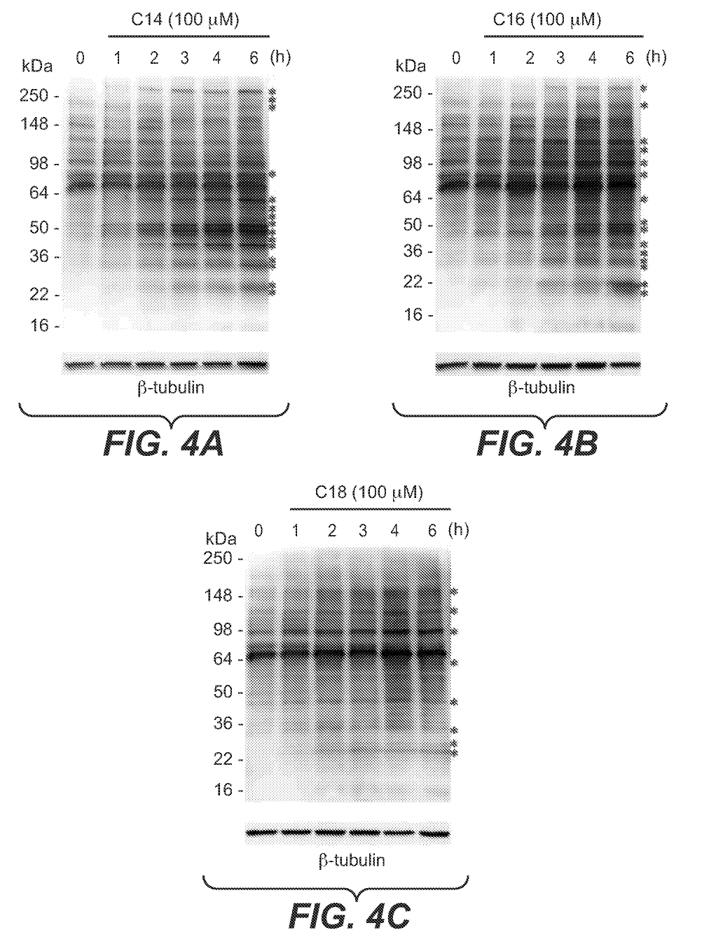

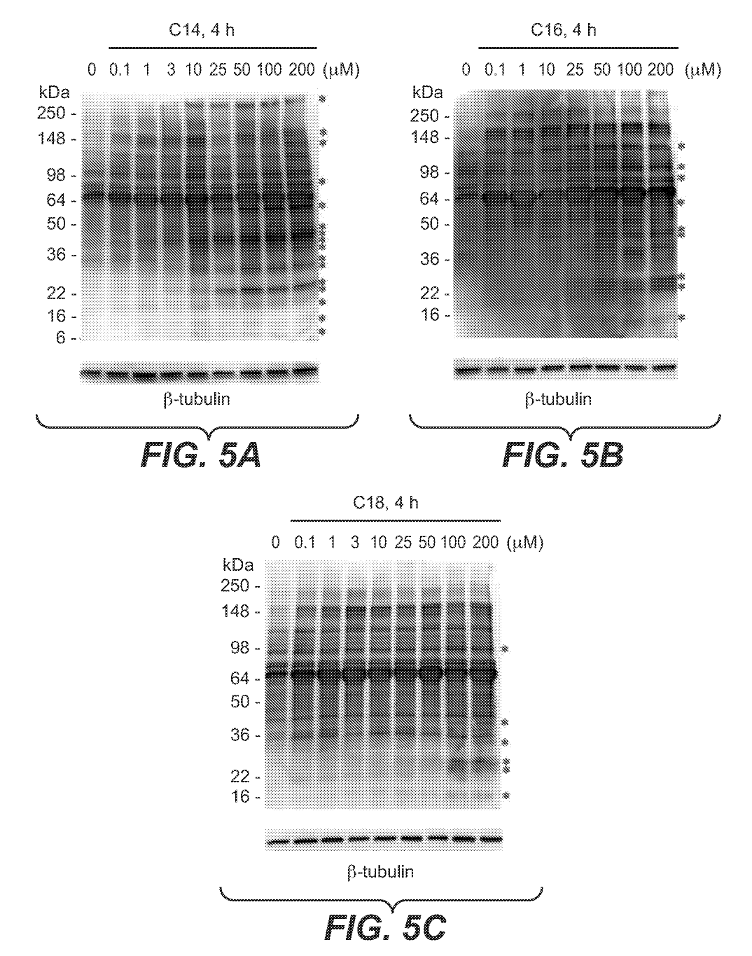

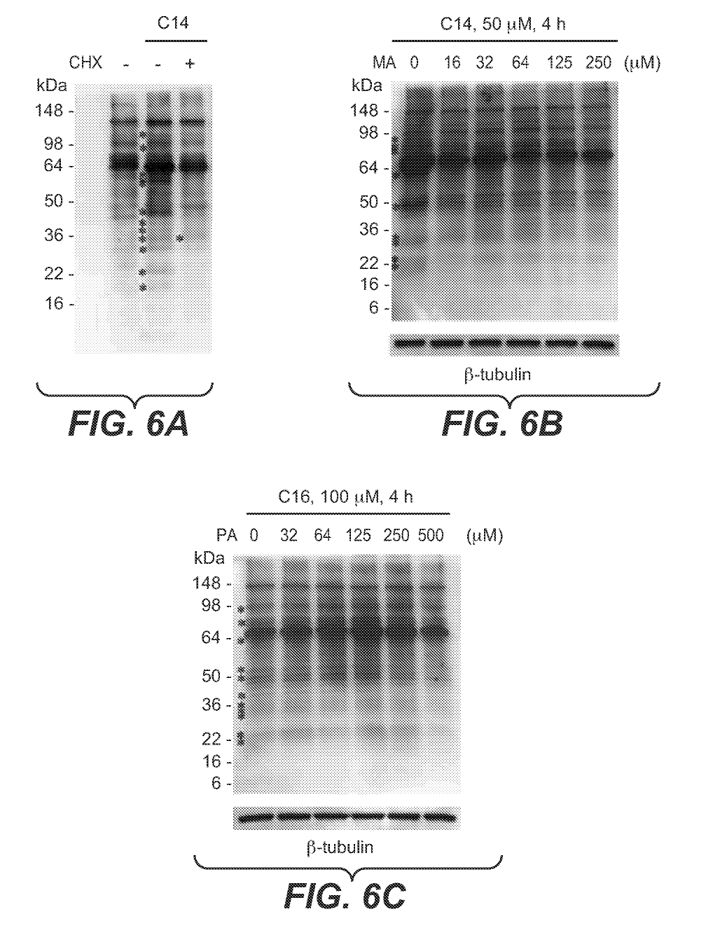

The ω-alkynyl fatty-acyls were metabolically incorporated onto cellular proteins in a time- and dose-dependent manner. Treatment of MDCK cells with C14, C16 or C18 fatty-acyls (100 μM) shows a time-dependent increase in the levels of labeled protein bands within 6 h (see, FIG. 4). In a similar fashion, treatment with increasing concentration of C14, C16 or C18 fatty-acyl shows a dose-dependent metabolic incorporation at 4 h (see, FIG. 5), indicating that labeling with ω-alkynyl fatty-acyls is dependent on active cellular metabolism. Because protein N-myristoylation is a co-translational event, inventor showed that treatment with the protein synthesis inhibitor cycloheximide inhibits protein labeling with C14 (see, FIG. 6A). Furthermore, competition experiments with myristic and palmitic acids demonstrate that the ω-alkynyl C14 and C16 fatty-acyls serve as specific probes for protein N-myristoylation and S-palmitoylation in cells, respectively (FIG. 6B, FIG. 6C). All together, these results illustrate that the ω-alkynyl fatty-acyls seem to be sufficiently uptaken and well tolerated by cultured cells, readily recognized by the biosynthetic machinery and efficiently incorporated onto cellular proteins.

Detection of Labeled Fatty-Acylated Proteins by Fluorescence Imaging

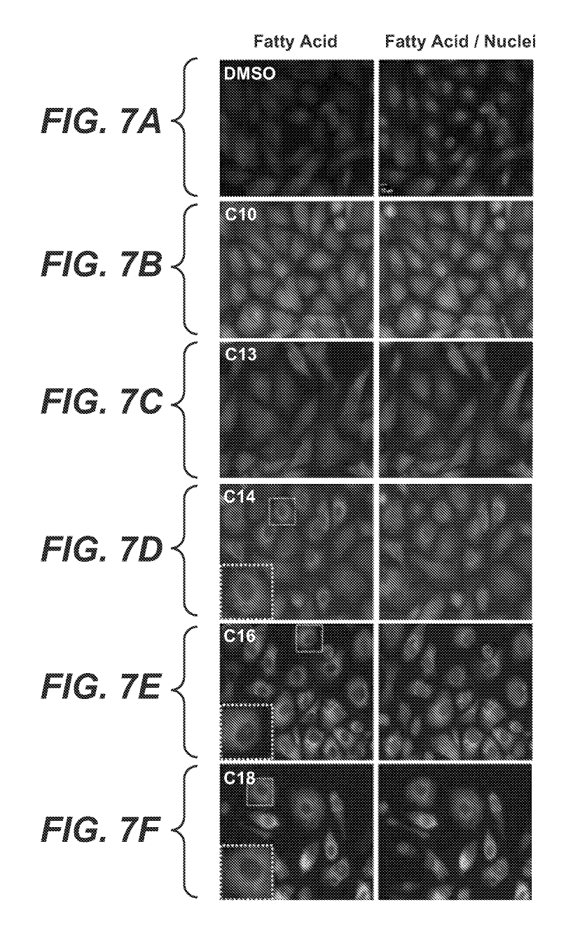

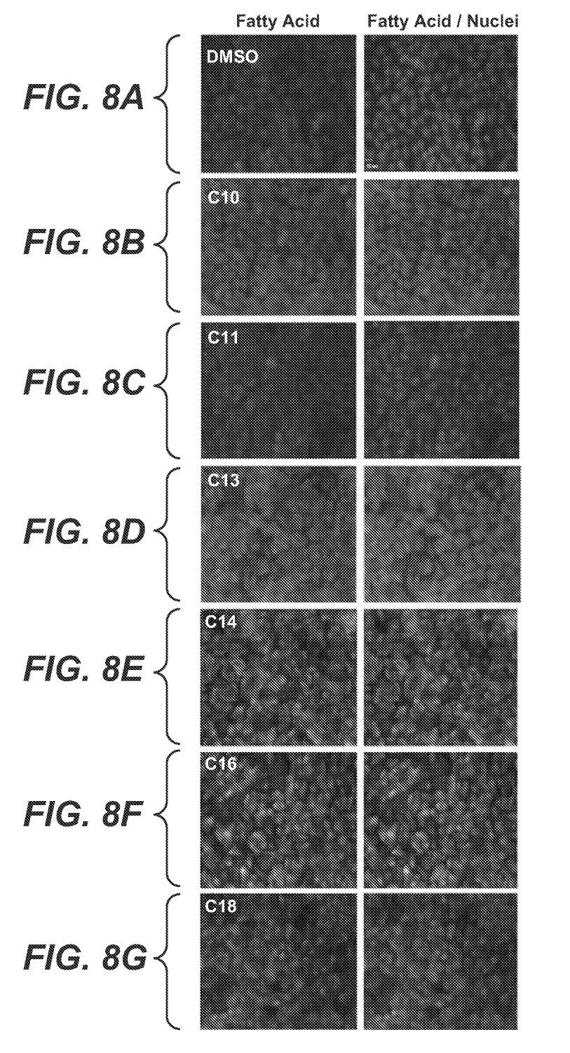

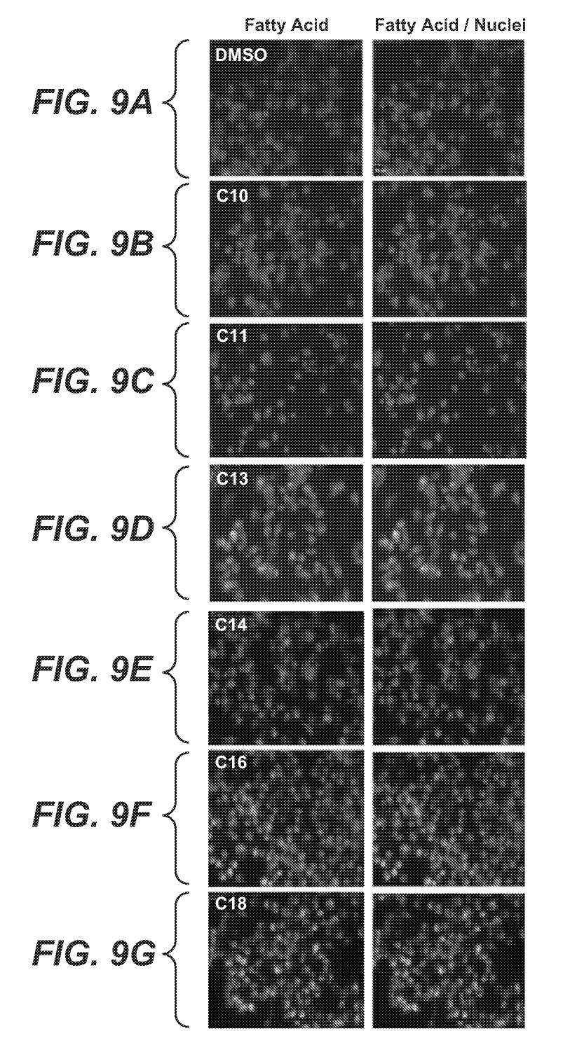

To demonstrate the broad utility of ω-alkynyl fatty-acyls for the in vivo detection of fatty-acylated proteins, we performed fluorescence microscopy to visualize cellular fatty-acylated proteins. PC3 prostate cancer cells were treated with vehicle (FIG. 2C) or the various ω-alkynyl fatty acid analogues, fixed and processed for click reaction with rhodamine azide or biotin azide, followed by streptavidin-conjugated Alexa488. A high fluorescence signal was observed in samples treated with ω-alkynyl fatty-acyls (FIG. 2D, FIG. 2E, FIG. 2F) compared to a minimal signal in DMSO-treated samples in PC3 cells FIG. 2C. A signal to background ratio was observed to be higher in samples processed with rhodamine azide compared to biotin azide, and this is due to endogenously biotinylated proteins that contribute to background. Fluorescence images show different subcellular distributions of the various ω-alkynyl fatty-acyls (see, description of FIG. 2C, FIG. 2D, FIG. 2E, FIG. 2F). A high fluorescent signal was observed in samples treated with ω-alkynyl fatty-acyls compared to a minimal signal in DMSO-treated samples in PC3 cells (FIG. 2(C-F) and FIG. 7(A-F)), mouse fibroblast L-cells (FIG. 8(A-G)) and RAW2647 macrophages (FIG. 9(A-G)). The signal to background ratio was typically higher in samples processed with rhodamine azide compared to biotin azide, and this is due to endogenously biotinylated proteins that contribute to background. The fluorescent images clearly show different subcellular distributions of the various ω-alkynyl fatty acids. Interestingly, confocal microscopy images (FIG. 2G, FIG. 2H, FIG. 2I) show that the C14, C16, and C18 fatty-acyl probes are distributed in a punctuate pattern outside the nucleus, localize in vesicular structures in the cytoplasm and label the plasma membrane and membrane ruffles. PC3 cells that are undergoing cell division and are labeled with C16 ω-alkynyl fatty acyl in addition to a tubulin marker were monitored by imaging (FIG. 2J, FIG. 2K, FIG. 2L). Metaphase cells show a distinct distribution of C16-labeled proteins at the plasma membrane and in dense structures around the spindle and throughout the body (FIG. 2K). Interestingly, during cytokinesis, C16-labeled proteins concentrate at the cleavage furrow (see arrow), the site of cell division (FIG. 2L).

### Example 2

### Synthesis of Compounds

General Procedures:

NMR spectra were recorded on a Varian 400 spectrometer using a ¹H or ¹³C solvent peak as internal reference (7.26 ppm for CHCl₃ and the CDCl₃ triplet at 77.26 ppm). Electrospray ionization (ESI) mass spectra (MS) were obtained on an Agilent API100 Perkin-Elmer SCIEX single quadrupole mass spectrometer at 4000 V emitter voltage in either positive- or negative-ion mode. Analytical thin-layer chromatography was performed with 0.25 mm E. Merck silica gel plates (60F-254) and visualized by dipping in a solution of KMnO₄ and heated. E. Merck silica gel 60 (particle size 0.040-0.063 mm) was used for column chromatography. All chemicals were obtained from Sigma Aldrich and used as received. Solvents used were of highest commercial grade available. Reactions were performed under inert atmosphere (N₂) with dry solvents under anhydrous conditions, unless otherwise indicated. Abbreviations used are: s (singlet), d (doublet), t (triplet), m (multiplet). Certain ω-alkynl fatty-acyls were commercially obtained as follows: compounds 1, 2, 6 (Sigma-Aldrich) and 3 (Otava Ltd., ON) (see FIG. 1b).

### Synthesis of Representative Examples of Compounds of Formula I

### 15-Hexadecyn-1-oic acid (5)

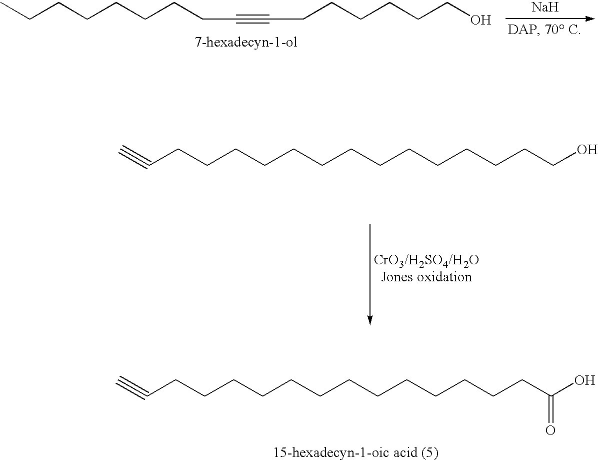

To NaH (60% in mineral oil, 720 mg, 17 mmol, washed twice with hexanes under N₂) was added diamino propane (DAP) (15 ml). The mixture was stirred in an oil bath at a constant temperature of 70° C. Evolution of gas was observed after 10 min and the solution turned brown after 1 h. The flask was cooled down to room temperature, and a solution of 7-hexadecyn-1-ol (512 mg, 2.15 mmol) dissolved in DAP (4 ml) was added. The mixture was stirred at 55° C. overnight during which it turned black. The flask was cooled down to room temperature, carefully hydrolyzed with ice-cold water, acidified with aqueous 10% HCl, and extracted three times with hexane (3×100 ml). The combined aqueous layers were extracted one more time with hexane, the combined organic layers were washed with saturated aqueous sodium bicarbonate and brine, dried with Na₂SO₄ and evaporated under vacuum. The crude yellow-brown product (˜0.5 g) was converted directly to the acid as described below.

To a solution of 15-hexadecyn-1-ol (150 mg, 0.63 mmol) in 20 ml acetone was added dropwise a solution of Jones' reagent until the characteristic deep orange red color persisted. After stirring for 5 mins, 2-propanol was added to neutralize the excess reagent until the color turned light green. The chromium salts were filtered, the acetone was evaporated, and the residue was dissolved in ethyl acetate and washed four times with 0.01 N HCl, dried with sodium sulfate and evaporated. The crude product was chromatographed (CH₂Cl₂, then hexane/EtOAc (4:1)) and recrystallized in hexane at −18° C. to yield a white solid (5) (140 mg, 88%). ¹H NMR (400 MHz, CDCl3) δ 2.35 (t, J=7.5, 2H), 2.18 (dt, J=2.6, 7.1, 2H), 1.94 (t, J=2.6, 1H), 1.69-1.57 (m, 2H), 1.57-1.46 (m, 2H), 1.26 (s, 18H). ¹³C NMR (101 MHz, CDCl3) δ 180.15, 85.05, 68.24, 34.23, 29.79, 29.71, 29.64, 29.45, 29.32, 29.27, 28.98, 28.71, 24.89, 18.61. MS (ESI+): m/z 253.4 (M+H)⁺.

### Synthesis of 13-Tetradecyn-1-oic acid (4)

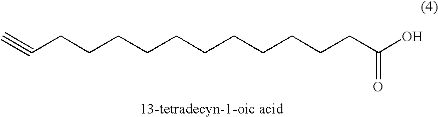

Compound 4 was prepared following the synthetic procedures described above to prepare compound 5, with the modification that 3-tetradecyn-1-ol as starting material: ¹H NMR (400 MHz, CDCl3) δ 2.35 (t, J=7.5, 2H), 2.18 (dt, J=2.6, 7.1, 2H), 1.94 (t, J=2.6, 1H), 1.69-1.57 (m, 2H), 1.57-1.46 (m, 2H), 1.27 (s, 14H). ¹³C NMR (101 MHz, CDCl3) δ 180.39, 85.02, 68.26, 34.27, 29.71, 29.67, 29.60, 29.44, 29.30, 29.25, 28.96, 28.69, 24.87, 18.61. MS (ESI−): m/z 223.4 (M−H)⁻.

### Synthesis of a biotin azide labeling group: N-(3-azidopropyl)-5-((3aS,4S,6aR)-2-oxohexahydro-1H-thieno[3,4-d]imidazol-4-yl)pentanamide (8)

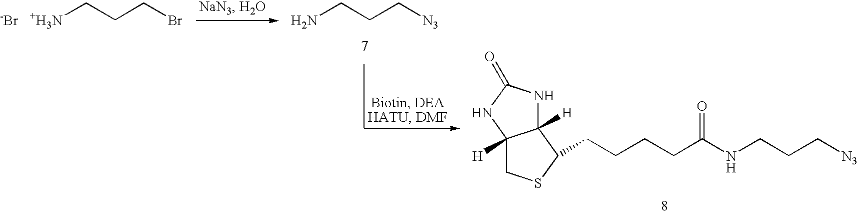

Synthesis of 3-Azido-propylamine (7): 3-bromopropylamine hydrobromide (9.76 g, 44.6 mmol) and sodium azide (6.19 g, 95.3 mmol) were dissolved in water (80 ml). The resulting solution was heated overnight at 80° C. After cooling to room temperature, about 50 ml of the water was evaporated under vacuum with gentle heating (˜50° C.), and the remaining mixture was stirred with 5% NaOH (20 ml) for 3 h at room temperature and then extracted with toluene (2×25 ml). An additional 40 ml of 5% NaOH was added to the aqueous phase, and further extraction with toluene was performed (4×25 ml). The combined organic extracts were dried over Na₂SO₄, filtered and evaporated under vacuum (˜40° C.) to yield 47 g of solution. The retained solution was found to contain 3.2 mol % of 3-azido-propylamine NMR integration, corresponding to 3.2% by weight (1.5 g) of the desired product (34% yield). The yellow product was used without further purification: ¹H NMR (400 MHz, CDCl3) δ 3.38 (t, J=6.7, 2H), 2.81 (t, J=6.8, 2H), 1.73 (p, J=6.8, 2H), 1.52 (s, 2H).

Synthesis of Biotin azide (8): To a solution of d-(+)-biotin (200 mg, 0.82 mmol), diisopropylethylamine (212 mg, 1.64 mmol), HATU (622 mg, 1.64 mmol) in DMF (10 ml) was added 7 (164 mg, 1.64 mmol), and the reaction allowed to stir overnight at room temperature. The reaction mixture was evaporated under vacuum and the residue purified by reverse phase chromatography to afford 8 (88 mg, 33% yield) as a white solid: ¹H NMR (400 MHz, DMSO) δ 7.80 (t, J=5.4, 1H), 6.39 (s, 1H), 6.33 (s, 1H), 4.36-4.22 (m, 1H), 4.17-4.07 (m, 1H), 3.34 (t, J=6.8, 2H), 3.17-2.99 (m, 3H), 2.82 (dd, J=12.4, 5.1, 1H), 2.58 (d, J=12.4, 1H), 2.06 (t, J=7.4, 2H), 1.73-1.56 (m, 3H), 1.56-1.39 (m, 3H), 1.33 (m, 2H). ¹³C NMR (101 MHz, DMSO) δ 172.01, 162.64, 61.00, 59.16, 55.35, 48.41, 35.71, 35.15, 28.43, 28.15, 25.20. MS (ESI+): m/z 327.1 (M+H)⁺.

### Example 3

### Biochemical Methods

Cell culture: Raw 264.7 macrophages (ATCC # CCL-2278), were grown in high glucose Dulbecco's Modified Eagle's Medium (DMEM) supplemented with 10% fetal bovine serum (FBS) and glutamax (2 mM). MDCK (canine kidney epithelial cells, ATCC # CCL-34) were grown in DMEM media supplemented with 10% FBS (ATCC #30-2003). PC-3 cells (ATCC # CRL-1435) were grown in F-12K Medium (ATCC #30-2004) supplemented with 10% FBS. Mouse L-cells (ATCC # CRL-2648) were cultured in DMEM media supplemented with 10% FBS (ATCC #30-2002). All cells used were incubated in a 5% CO₂ humidified incubator at 37° C. for 24 h before any experiment.

Labeling and detection of lipoproteins in cell extracts: The ω-alkynl fatty-acyl compounds used for the examples were dissolved in DMSO to generate 50 mM stock solutions, and were stored at −80° C. Before cell treatment, the analogs were dissolved in DMEM serum-free media supplemented with 5% BSA (fatty acid-free—SIGMA EC232-936-2) and glutamax (for Raw and MDCK cell lines) at a final concentration of 100 μM. The fatty acid-media solutions were sonicated for 15 minutes at room temperature and then allowed to pre-complex for 15 min at RT.

Cells were seeded with complete media onto 6-well plates (8×10⁵ cells/2 ml/well). They were incubated for 24 h before the treatment. Then the growth medium was removed, cells washed once with PBS and 2 mL of the ω-alkynyl fatty-acyls containing media was added to cells and incubated at 37° C. in a 5% CO₂ humidified incubator. After 24 hours, the cells were washed three times with cold PBS and cell extracts were prepared by resuspending the cells in 400 μL of lysis buffer (1% Nonidet P-40/150 mM NaCl/protease and phosphatase inhibitor/100 mM sodium phosphate, pH 7.5). To obtain a final proteome concentration of 2 mg/ml (protein concentration determined by BCA kit) cell lysates were concentrated by centrifugation for 15 minutes at 14,000 rpm at 4° C. with the Centrifugal Ultrafiltration Devices (Pall centrifugal devices MWCO 3K, Nanosep device, cat # P/N ODOO3C34). Protein extracts were then subjected to the probe labeling reaction in 25 μL volume, for 1 h at RT (room temperature), at final concentrations of the following reagents (See, Speers, A. E. & Cravatt, B. F. Profiling enzyme activities in vivo using click chemistry methods. Chem Biol 11, 535-46 (2004); Hsu, T. L. et al. Alkynyl sugar analogs for the labeling and visualization of glycoconjugates in cells. Proc Natl Acad Sci USA 104, 2614-9 (2007)): 0.1 mM biotin-azide, 1 mM Tris (2-carboxyethyl)phosphine hydrochloride (TCEP, Sigma-Aldrich) dissolved in water, 0.2 mM Tris[(1-benzyl-1H-1,2,3-triazol-4-yl)methyl]amine (TBTA, Sigma-Aldrich) dissolved in DMSO/t-butanol (20%/80%) and 1 mM CuSO₄ in PBS. The order of addition of the reagents to the protein extracts is important for the reaction and has to be followed as described above.

Western blotting: Labeled protein lysates were resolved by SDS page using a 4-20% Tris-glycine gel (1 h 10 min at 180V). For immunoblotting of biotin-labeled proteins after electrophoresis, proteins were transferred onto a nitrocellulose membrane, which was blocked with PBS, 0.1% Tween-20 [PBST] and 5% non-fat dried milk for 2 h at RT or overnight at 4° C. The membrane was washed three times with PBST (5 minutes each), and incubated with streptavidin-horseradish peroxidase (Invitrogen Zymed #43-4323, 1:1250 in PBST) for 1 h at RT. The membrane was washed with PBST three times (10 min each) and developed using enhanced chemiluminescence according to manufacturer's recommendation (Amersham Biosciences). For the hydroxylamine-sensitive assay, following the transfer of proteins to nitrocellulose membranes, the membranes were incubated 65 to 72 h at RT with PBST and 5% NH₂OH (Sigma-Aldrich). After the hydroxylamine treatment, the membranes were blocked with 5% non-fat dried milk for 2 h at RT or overnight at 4° C. and analyzed by streptavidin blot as described above. To demonstrate equal levels of protein loading, streptavidin blots were stripped with Pierce stripping buffer for 15 min at RT and reprobed with an anti-β-tubulin HRP antibody and developed with enhanced chemiluminescence.

Fluorescence microscopy: Cells were seeded onto 12-well plates (4×10⁵ cells/well) containing coverslips and incubated for 24 h before treatment. The growth medium was removed and cells were washed once with PBS before adding 1 mL of medium containing the co-alkynyl fatty acid at the indicated concentration. After 24-48 h incubation at 37° C./5% CO₂, cells were washed three times with PBS to remove excess probe (ω-alkynyl fatty acid) and fixed with 4% paraformaldehyde (PFA) for 10 min at RT. Cells were then permeabilized with PBS/0.1% triton X-100 for 1-2 min at RT, washed extensively with the following reagents: 0.1 mM biotin-azide or rhodamine-azide, 1 mM Tris (2-carboxyethyl)phosphine hydrochloride (TCEP) dissolved in water, and 1 mM CuSO₄ in PBS at RT for 1 h. The labeled cells were rinsed extensively with PBS and blocked in PBS/5% BSA for 45 min at RT. Cells were stained with streptavidin-conjugated AlexaFluor 488 (Invitrogen cat # S32354, 1:500) in PBS/5% BSA for 45 min at RT and nuclei were stained with Hoechst 33342 (MP # H21492; 1:10,000 in PBS) for 10 min at RT. For labeling with rhodamine-azide, cells were directly stained with Hoechst. For tubulin staining, cells were fixed in pre-cooled methanol at −20° C. for 5-10 min and processed for the click reaction as described above followed by staining with anti-tubulin antibody and the appropriate secondary Alexa488 conjugate antibody. Fluorescent images were captured on an inverted Zeiss AX10 microscope equipped with a CoolSnap CCD camera (Roper Scientific) and images were analyzed with Slidebook 4.1 software (Intelligent Imaging Innovation). Z-sections were acquired with 0.3 μm spacing. An average of 50-70 z-sections were acquired per image.

We claim:

1. A method of detecting a fatty-acylated substrate comprising:
i. incubating a fatty acyl of Formula I with an animal cell 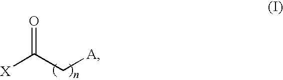
wherein in Formula I the subscript n is an integer from 6 to 15, the symbol A represents an ethynyl group and the symbol X represents —OH or —SCoA, wherein said animal cell comprises a substrate and at least one enzyme capable of attaching I to the substrate, to produce a fatty-acylated substrate;
ii. combining the fatty-acylated substrate from step (i) with an azido tagged labeling group wherein the azido tag undergoes a [3+2] cycloaddition reaction with the A group on the fatty-acylated substrate to produce a labeled fatty-acylated substrate; and
iii. detecting the labeling group on the fatty-acylated substrate in vivo in an animal cell by fluorescence imaging; and thereby detecting the fatty-acylated substrate.

2. The method of claim 1, wherein said method is performed using a mammalian cell.

3. The method of claim 2, wherein said cell is a cancer cell.

4. The method of claim 1, wherein said enzyme is acyltransferase.

5. The method of claim 4, wherein said enzyme is selected from the group consisting of N-myristoyltransferase, S-acyltransferase and S-palmitoyltransferase.

6. The method of claim 1, wherein in Formula I the subscript n is an integer from 7 to 14.

7. The method of claim 6, wherein the subscript n is an integer selected from the group consisting of 7, 8, 10, 11 and 13.

8. The method of claim 7, wherein the subscript n is the integer 11 or 13.

9. The method of claim 1, wherein X is —OH.

10. The method of claim 1, wherein X is —SCoA.

11. The method of claim 1, wherein said substrate is a protein or polypeptide.

12. The method of claim 1, wherein said labeling group is selected from the group consisting of a label enzyme and a fluorescent labeling group.

13. The method of claim 12, wherein said labeling group is rhodamine azide.

14. The method of claim 1, wherein said labeling group comprises a member of a binding pair.

15. The method of claim 14, wherein between steps (ii) and (iii) is a step of treating the labeled fatty-acylated substrate produced from step (ii) with a detectable labeling group comprising the complementary member of said binding pair, and wherein said complementary member of said binding pair binds to the labeling group of said labeled fatty-acylated substrate produced from step (ii).

16. The method of claim 14, wherein said labeling group is biotin azide.

17. The method of claim 15, wherein said complementary member of said binding pair is streptavidin linked to a fluorophore.

18. The method of claim 17, wherein said complementary member of said binding pair is streptavidin linked to AlexaFluor 488.

19. Use of a fatty-acyl compound of Formula I in an in vivo assay in an animal cell for the detection of fatty-acylation of a protein or polypeptide, 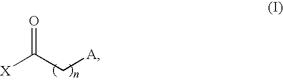
wherein in Formula I the subscript n is an integer from 6 to 15, the symbol A represents an ethynyl group and the symbol X represents —OH or —ScoA, and wherein the detection occurs in an in vivo setting.

20. The use of claim 19, wherein n is the integer 11 or 13.
